(19) United States
(12) Patent Application Publication
(10) Pub. No.: US 2025/0109383 A1
(43) Pub. Date: Apr. 3, 2025

(54) METHOD FOR PRODUCING ADIPOSE MODEL THROUGH ENVIRONMENTAL CONTROL AND ADIPOSE MODEL CREATED THEREBY

(71) Applicants: POSTECH Research and Business Development Foundation, Pohang-si, Gyeongsangbuk-do (KR); PUSAN NATIONAL UNIVERSITY INDUSTRY-UNIVERSITY COOPERATION FOUNDATION, Busan (KR)

(72) Inventors: Dong-Woo CHO, Nam-gu (KR); Byoung Soo KIM, Yangsan-si (KR); Won-Woo CHO, Nam-gu (KR); Min Jun AHN, Nam-gu (KR)

(73) Assignees: POSTECH Research and Business Development Foundation, Pohang-si, Gyeongsangbu (KR); PUSAN NATIONAL UNIVERSITY INDUSTRY-UNIVERSITY COOPERATION FOUNDATION, Busan (KR)

(21) Appl. No.: 18/834,936

(22) PCT Filed: Jan. 4, 2023
(86) PCT No.: PCT/KR2023/000154

§ 371 Date: Jul. 31, 2024

(30) Foreign Application Priority Data
Mar. 8, 2022 (KR) 10-2022-0029565

(51) Int. Cl. C12N 5/077; B33Y 10/00; B33Y 80/00

(52) U.S. Cl. CPC C12N 5/0653; B33Y 10/00; B33Y 80/00; C12N 2513/00; C12N 2533/74

(57) ABSTRACT

The present invention relates to a method for producing an adipose model through environmental control and an adipose model created using same, the method comprising the steps of: providing a first bioink, which is cell-unfriendly; creating a bath suspension with a predetermined volume by using the first bioink; providing a second bioink comprising preadipocytes; 3Dprinting the second bioink in the bath suspension so as to produce an adipose model; and culturing the bath suspension in which the adipose model is disposed. The method for producing an adipose model through environmental control and the adipose model created using same, according to the present disclosure, form environments for a 3D-printed adipose model, and limit the proliferation area of adipose cells and induce the differentiation thereof, and thus can maximize similarity with actual adipose tissues.

### TECHNICAL FIELD

The research related to the disclosure was conducted with the support of Nano-Materials Technology Development (R&D) (Project title: Development of 3D nano/micro cell printing-based materials and manufacturing technology for multi-scale biomimetic artificial blood vessels and complex muscle tissue, and Project No.: 1711127593) under the supervision of the Ministry of Science and information and communications technology (ICT), and the support of Personal Basic Research (Project title: Development of the next-generation 3D high-speed tissue printing platform for permanent alopecia treatment and hair regrowth, and Project No.: 1711157589) under the supervision of the Ministry of Science and ICT.

The disclosure relates to an adipose model producing method based on environmental control, in which an adipose model is produced by 3D printing under control of environment to increase similarity to actual tissue, and an adipose model produced by the same.

### Background Art

Adipose tissue is representative soft tissue, and densely contains adipocytes mostly filled with lipid droplets.

Recently, it has been discovered that the adipose tissue not only serves as an energy storage for storing energy in the form of lipids but is also the largest endocrine organ in a human body as shown in FIG. 1. In particular, it has been discovered that the adipose tissue is closely related to the metabolism of other organs by secreting various hormones and signaling substances (cytokines) called adipokines.

Meanwhile, in relation to the adipose tissue, Korean patent publication No. 10-2021-0077327 is disclosed. However, such a related art for the reproduction of the adipose tissue has problems in that mimetics created by reproducing the adipose tissue do not maintain their shapes but rapidly contract, thereby causing a hypofunction.

### DISCLOSURE

### Technical Problem

An aspect of the disclosure is to provide an adipose model producing method based on environmental control, which can maximize structural and functional similarities to actual adipose tissue upon conventional adipose cell reproduction, and an adipose model produced by the same.

### Technical Solution

According to an embodiment of the disclosure, there may be provided a method of producing an adipose model through environmental control, the method including: preparing a cell-unfriendly first bio-ink; forming a bath suspension with the first bio-ink to have a predetermined volume; preparing a second bio-ink that contains preadipocytes; producing the adipose model by three-dimensionally printing the second bio-ink in the bath suspension; and culturing the bath suspension with the adipose model provided therein.

Meanwhile, the first bio-ink may contain no cell-binding motifs.

Further, the first bio-ink may contain an adipose derived extracellular matrix.

Further, the first bio-ink may contain a substance to prevent the preadipocytes of the adipose model from infiltrating into the bath suspension during proliferation and differentiation.

Meanwhile, the first bio-ink may contain alginate.

Meanwhile, the first bio-ink may contain 1 to 2% weight/volume of the adipose derived extracellular matrix.

Further, the first bio-ink may contain 1 to 2% weight/volume of the alginate.

Meanwhile, the bath suspension has properties that exhibit the Bingham plastic behavior.

Meanwhile, the preadipocytes contained in the second bio-ink have a concentration of 1×10⁶ to 1×10⁸ cells/mL.

In addition, there may be provided an adipose model produced using an environmentally-controlled bath suspension, by performing: preparing a cell-unfriendly first bio-ink; forming a bath suspension with the first bio-ink to have a predetermined volume; preparing a second bio-ink that contains preadipocytes; producing the adipose model by three-dimensionally printing the second bio-ink in the bath suspension; and culturing the bath suspension with the adipose model provided therein.

### Advantageous Effects

According to the disclosure, an adipose model producing method based on environmental control, and an adipose model produced by the same have effects on maximizing similarities to actual adipose tissue by controlling environment of the produced adipose model.

### DESCRIPTION OF DRAWINGS

FIG. 1 is a conceptual view showing an anatomical structure of adipose tissue and body's internal organs interacting with the adipose tissue.

FIG. 2 is a view showing limitations of a structure created by mimicking adipose tissue according to the related art.

FIG. 3 is a flowchart of a method of producing an adipose model through environmental control according to an embodiment of the disclosure.

FIG. 4 is a conceptual view showing an adipose model based on a method of producing an adipose model through environmental control according to an embodiment of the disclosure.

FIG. 5 is a conceptual view showing proliferative behavior of cells based on a method of producing an adipose model through environmental control according to an embodiment of the disclosure.

FIG. 6 shows results of evaluating the viscoelastic properties of a first bio-ink.

FIG. 7 is a view showing results of crosslinking by calcium according to the concentration of alginate contained in a first bio-ink.

FIG. 8 shows results of preadipocyte proliferation according to the concentration of a first bio-ink.

FIG. 9 shows experimental results for comparing adipose tissue created by various conventional methods and a method of producing an adipose model through environmental control according to an embodiment of the disclosure.

FIG. 10 shows function evaluation results of an adipose model created by a method of producing the adipose mode through environmental control according to an embodiment of the disclosure.

### MODE FOR INVENTION

Below, an adipose model producing method based on environmental control, and an adipose model created by the same will be described in detail with reference to the accompanying drawings. In the following description, the terms of components may be referred to as other terms in the art. However, as long as the components have functional similarity and identity therebetween, they may be considered as equivalent components even in alternative embodiments. Further, reference numerals assigned to the components are provided for the convenience of description. However, content indicated by the reference numerals in the drawings do not limit the components to the scope shown in the drawings. Similarly, as long as the configurations in the drawings have functional similarity and identity therebetween, they may be considered as equivalent configurations even in alternative embodiments. Further, descriptions will be omitted for components that should be involved naturally at the level of those skilled in the art.

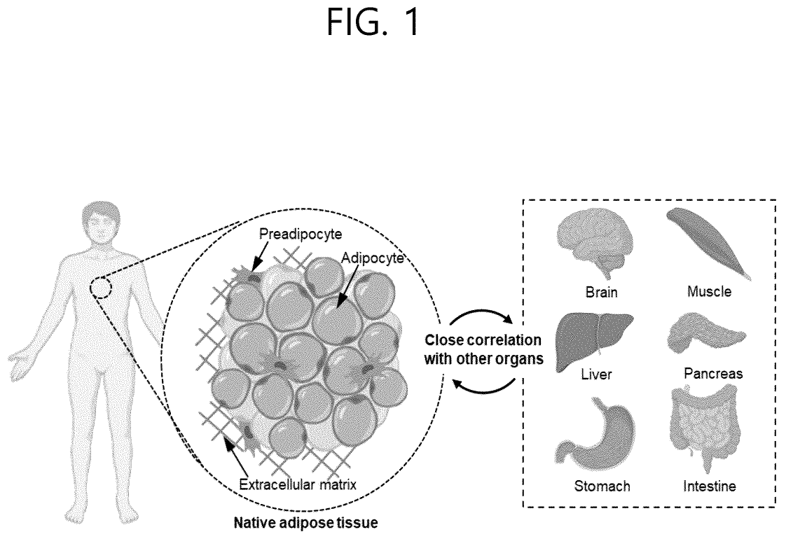

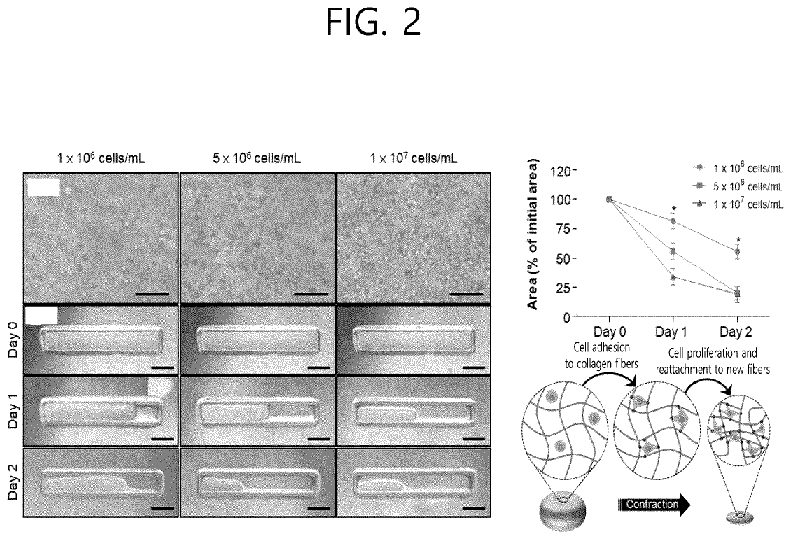

As described above with reference to FIG. 1 in connection with the background art, various methods of mimicking and creating adipose tissue have been disclosed as shown in FIG. 2 with the newly discovered functions of adipose tissue.

FIG. 2 is a view showing limitations of a structure created by mimicking adipose tissue according to the related art.

Referring to FIG. 2, a method of producing bio-ink, which contains preadipocytes, by direct printing has been widely used. In this case, adipose mimetic tissue created with a first bio-ink always contracts regardless of difference in the concentration of preadipocytes when surrounding environment is not controlled.

Referring to FIG. 2, the first bio-ink was printed at concentrations of 1×10⁶, 5×10⁶, and 1×10⁷ cells/mL (the top left in FIG. 2), and contracted when cultured for two days (see the bottom left and the top right in FIG. 2). This is because the preadipocytes adhered to collagen fibers in an adipose derived extracellular matrix (AdECM) pull the fiber during proliferation and migration. As the number of cells increases, the bio-ink is unable to withstand this pulling force and contracts (see the bottom right in FIG. 2). Such contraction occurs rapidly, and the rapid contraction of the bio-ink causes apoptosis and deterioration of adipocytes, thereby ultimately leading to an inferior tissue structure.

According to the disclosure, the environment of the preadipocytes to be printed is controlled to solve such a conventional problem.

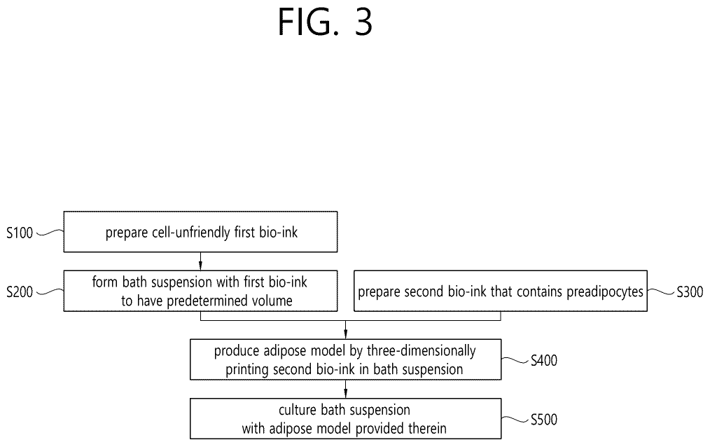

FIG. 3 is a flowchart of a method of producing an adipose model through environmental control according to an embodiment of the disclosure.

Referring to FIG. 3, the method of producing the adipose model through the environmental control according to an embodiment of the disclosure may include the steps of preparing a cell-unfriendly first bio-ink (S100), forming a bath suspension with the first bio-ink (S200), preparing a second bio-ink that contains preadipocytes (S300), producing an adipose model by three-dimensionally printing the second bio-ink in the bath suspension (S400), and culturing the bath suspension with the adipose model provided therein (S500).

The step S100 of preparing the cell-unfriendly first bio-ink corresponds to a step of preparing the first bio-ink to form the bath suspension subsequently. In this step, the first bio-ink is prepared with ingredients to provide an environment in which the adipocytes to be printed in the bath suspension later are prevented from infiltrating into the bath suspension.

The first bio-ink prepared in this step S100 may have cell-unfriendly properties. Specifically, the first bio-ink may include a biomaterial that has no cell-binding motifs. For example, the first bio-ink may contain alginate as the biomaterial that has no cell-binding motif. Such properties prevent the adipocytes to be printed later from infiltrating into the bath suspension formed with the first bio-ink.

Further, the first bio-ink may contain the adipose derived extracellular matrix (AdECM) for the differentiation and growth of the preadipocytes contained in the second bio-ink.

Meanwhile, the first bio-ink may be prepared to have the properties so that the second bio-ink extruded upon printing after nozzles for printing the second bio-ink advance into the bath suspension cannot infiltrated into the bath suspension. For example, the first bio-ink may be prepared to contain ingredients for the Bingham plastic behavior. In other words, the first bio-ink exhibits liquid behavior (loss modulus>storage modulus) under strong shearing force, and exhibits solid behavior (storage modulus>loss modulus) under weak shearing force. Here, the strong shearing force refers to a condition of when the first bio-ink is extruded by a 3D printer, and the first bio-ink under this condition needs to have the liquid behavior for smooth printing. On the other hand, the weak shearing force refers to a condition that the nozzles are not present or do not move in the bath suspension, and the printed first bio-ink under this condition needs to have the solid behavior for repelling the second bio-ink.

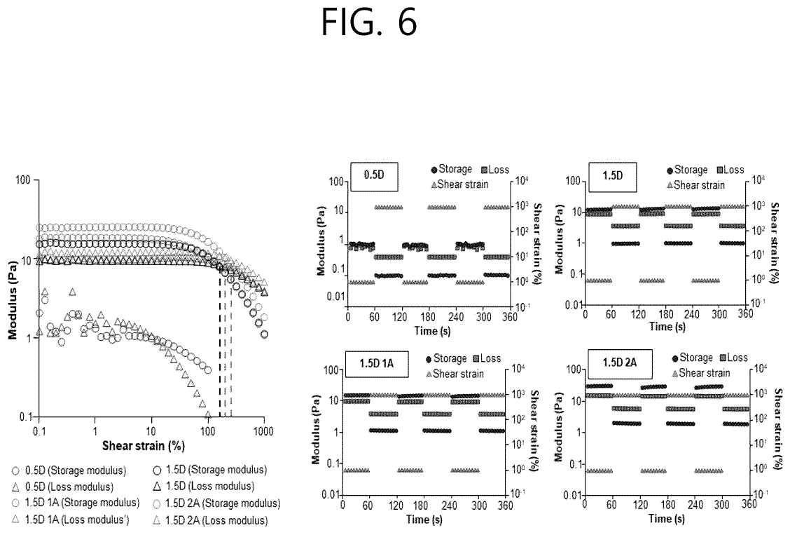

For example, the first bio-ink may contain 1 to 2% weight/volume of AdECM and 1 to 2% weight/volume of alginate. Preferably, the first bio-ink may contain 1.5% weight/volume of AdECM and 1.0% weight/volume of alginate. In this regard, the concentration of the first bio-ink will be described later with reference to FIGS. 6 to 8.

The step S200 of forming the bath suspension with the first bio-ink corresponds to a step of printing the first bio-ink prepared at an appropriate concentration as the bath suspension having a predetermined volume. In this step, the first bio-ink exhibits the Bingham plastic behavior, and thus behaves like a solid in the state that no external force is exerted after being smoothly printed.

The step S300 of preparing the second bio-ink that contains the preadipocytes refers to a step of preparing a material to produce the adipose model. In this step S300, the second bio-ink is prepared by encapsulating a high concentration of preadipocytes and the adipose derived extracellular matrix together. For example, the second bio-ink may contain 1×10⁶ to 1×10⁸ cells/mL of preadipocytes.

The step S400 of producing the adipose model by three-dimensionally printing the second bio-ink in the bath suspension may be performed by three-dimensionally printing the second bio-ink in the bath suspension formed with the first bio-ink. In this step S400, the printed adipose model may be surrounded with the first bio-ink in the bath suspension, thereby providing an environment where the adipose model is differentiated by the properties of the bath suspension.

The step S500 of culturing the bath suspension with the adipose model provided therein may be included. In this step S500, the adipose model may be differentiated through an adipogenic differentiation medium.

Meanwhile, the step of preparing the first bio-ink and the step of preparing the second bio-ink may be performed regardless of the foregoing order. However, the first bio-ink and the second bio-ink may be prepared before the printing.

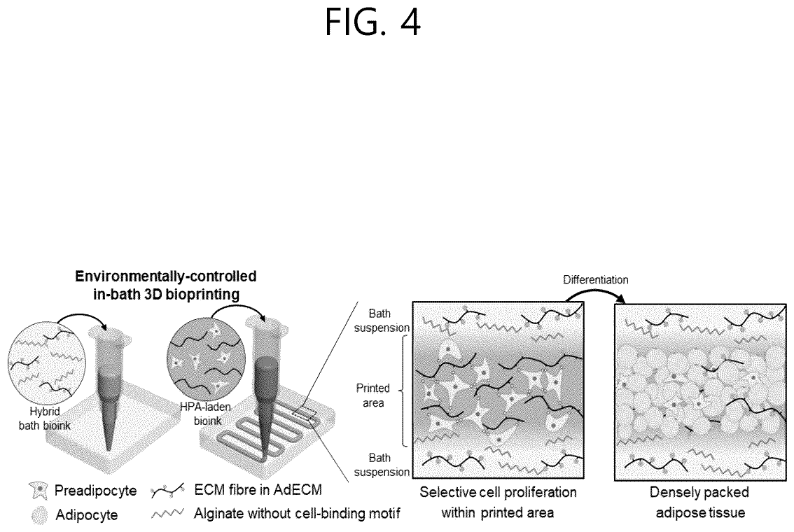

FIG. 4 is a conceptual view showing an adipose model based on a method of producing an adipose model through environmental control according to an embodiment of the disclosure.

Referring to FIG. 4, as described above, a first bio-ink 10 may contain cell-unfriendly alginate, and cell-friendly adipose derived extracellular matrix (AdECM), and be printed to form a bath suspension 100.

Then, the 3D printer is used to print a second bio-ink 20 into the bath suspension 100.

Ultimately, in an adipose model 1 produced by the method of producing the adipose model through the environmental control, the bath suspension 100 provides the cell-unfriendly environment, and thus the preadipocytes proliferate and differentiate within a limited area where the second bio-ink has been initially printed. In other words, the preadipocytes and the adipocytes remain without infiltrating into the bath suspension. Eventually, according to the disclosure, adipose tissue is densely packed with the adipocytes like the anatomical structure of actual adipose tissue.

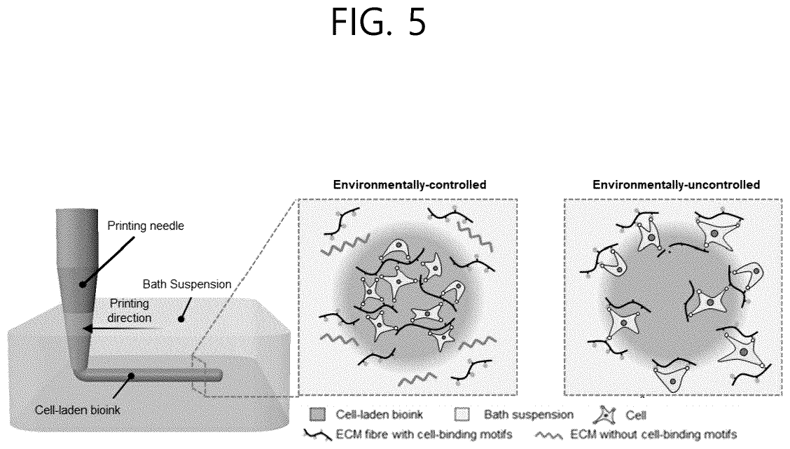

Below, the composition of the first bio-ink and the composition of the second bio-ink will be described in detail with reference to FIGS. 5 to 10.

FIG. 5 is a conceptual view showing proliferative behavior of cells based on a method of producing an adipose model through environmental control according to an embodiment of the disclosure.

Referring to FIG. 5, differentiation results of the adipose model are compared by performing the printing with different conditions of the bath suspension under the same printing condition.

The left box in FIG. 5 shows that the adipose model is environmentally-controlled according to the disclosure by including the cell-unfriendly substances in the bath suspension. In this case, the preadipocytes proliferate and differentiate densely in the area where the first bio-ink is printed, without infiltrating into the bath suspension.

On the other hand, the right box in FIG. 5 shows that the printed adipose model is environmentally-uncontrolled. In this case, the preadipocytes infiltrate into the bath suspension and become sparse while proliferating, and thus have poor similarity to the actual adipose tissue after differentiation. Therefore, the composition of the first bio-ink that forms the surrounding environment is more important than the composition of the second bio-ink that is three-dimensionally printed to mimic the adipose tissue.

FIG. 6 shows results of evaluating the viscoelastic properties of a first bio-ink.

As described above, when used to form the bath suspension, the first bio-ink is required to have the properties of allowing the second bio-ink to be smoothly printed in the bath suspension formed with the first bio-ink, and preventing the preadipocytes from leaving their locations after the adipose model is printed.

The properties of the bath suspension may vary depending on the concentrations of AdECM and alginate that constitute the first bio-ink.

To this end, viscoelastic behavior may be checked when the first bio-ink contains 0.5 or 1.5% weight/volume of AdECM and 1 or 2% weight/volume of alginate.

In other words, the viscoelastic properties were compared when the adipose model was produced using the second bio-ink within four bath suspensions formed as follows:
i) 0.5% (w/v) AdECM only: 0.5D, ii) 1.5% (w/v) AdECM only: 1.5D, iii) 1.5% (w/v) AdECM+1% (w/v) alginate: 1.5D 1A, and iv) 1.5% (w/v) AdECM+2% (w/v) alginate: 1.5D 2A

Referring to the left graph in FIG. 6, when the AdECM has a low concentration as in the condition i), the storage modulus and the loss modulus are not constant but vary irregularly under the weak shearing force. In other words, it was confirmed that the AdECM having the low concentration of the condition i) was not suitable because it behaved like a liquid even in a stationary state. When the AdECM having the concentrations of the conditions ii), iii) and iv) had clear storage and loss moduli for shear strain, its storage and loss moduli were the same as those based on the behavior of the Bingham plastic materials.

Referring to the right graphs in FIG. 6, when the bath suspensions were formed according to the concentrations and the printing was performed using the second bio-ink, test specimens were subjected to strong or weak shearing force at intervals of 60 seconds to test whether the printed adipose model maintained its structure well, thereby evaluating the capability of shear recovery of the materials. As a result, it was confirmed that the shear recovery properties had the characteristics of the Bingham plastic behavior in the cases of ii) iii) and iv) except the case of i) where the AdECM has the low concentration (0.5 (w/v)). Meanwhile, in this case, it was confirmed that shear recovery properties were observed even in the cases of (iii) and iv) where alginate was contained within 2%.

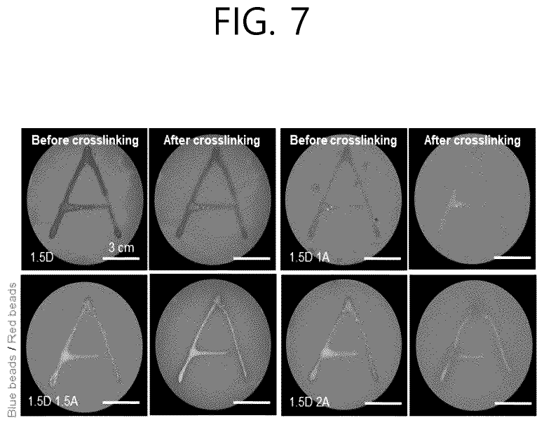

FIG. 7 is a view showing results of crosslinking by calcium according to the concentration of alginate contained in a first bio-ink.

Alginate contained in the first bio-ink is rapidly crosslinked by calcium ions (Ca²⁺), and the rapid crosslinking of the bath suspension is undesirable in terms of printing and maintaining the shape of the second bio-ink. To check the crosslinking tendency of the bath suspension, red fluorescent beads were mixed with the second bio-ink, blue beads were mixed with the first bio-ink, and the letter “A” was printed in the bath suspension. In this case, the composition of the first bio-ink was as follows.
1) 1.5% (w/v) AdECM only: 1.5D, 2) 1.5% (w/v) AdECM+1% (w/v) alginate: 1.5D 1A, 3) 1.5% (w/v) AdECM+1.5% (w/v) alginate: 1.5D 1.5A, and 4) 1.5% (w/v) AdECM+2% (w/v) alginate: 1.5D 2A

Referring to FIG. 7, when the four bath suspensions formed as above were crosslinked by injecting calcium ions thereinto, it was confirmed that the printed shape of the second bio-ink was seriously distorted in the case of alginate having the concentration of 2% weight/volume. Ultimately, it is preferable that the concentration of alginate in the first bio-ink is less than 2% weight/volume.

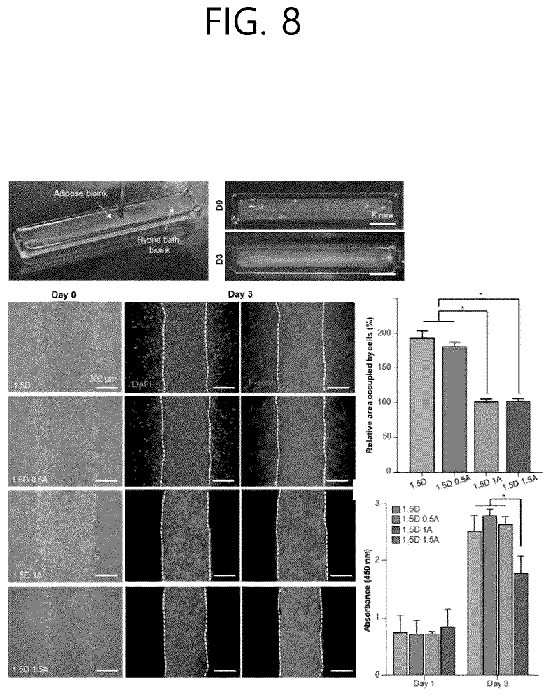

FIG. 8 shows results of preadipocyte proliferation according to the concentration of a first bio-ink.

Referring to the top in FIG. 8, to determine the concentration of the first bio-ink appropriate for controlling the proliferation of cells, the first bio-ink was filled into a rectangular structure of 15 mm×5 mm×1 mm, and the second bio-ink with the preadipocytes encapsulated therein was printed in the first bio-ink along a straight line for the test.

The bottom left in FIG. 8 shows the differentiation results of the adipose model under the conditions that the prepared first bio-ink contains the AdECM having a concentration of 1.5% weight/volume and alginate having concentrations of 0, 0.5, 1, and 1.5% weight/volume. As a result of DAPI and F-actin staining, it was confirmed that cells escaped from the printed location toward the bath suspension after 3 days of culture in the case of 0.5% weight/volume of alginate as in the case of no alginate. On the other hand, it was confirmed that the cells were densely packed in the printed location without escaping out of the printed location when 1 and 1.5% weight/volume of alginate was contained in the first bio-ink.

Referring to the top right in FIG. 8, after culturing the adipose tissue for 3 days in each condition, the number of cells in the printed location was counted to evaluate how many cells were not packed in the printed position but escaped toward the bath suspension. This quantitative evaluation supported the results of fluorescence microscopy.

Referring to the bottom right in FIG. 8, experiment results of a cell counting kit-8 (CCK-8) to evaluate a degree of cell proliferation in the respective conditions are shown. Experimental groups other than that under the condition of 1.5D 1.5A (1.5% weight/volume of AdECM, and 1.5% weight/volume of alginate) showed similar degrees of cell proliferation, but the experimental group under the condition of 1.5D 1.5A showed significantly less proliferation. In conclusion, the high concentration of alginate around the cells has a negative effect on the proliferation of the cells, and it is thus preferable that the first bio-ink contains about 1% weight/volume of alginate.

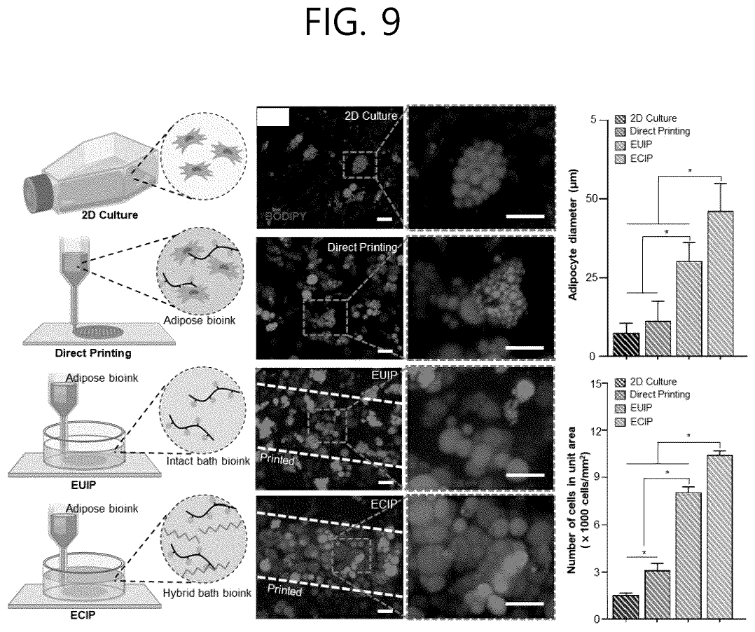

FIG. 9 shows experimental results for comparing adipose tissue created by various conventional methods and a method of producing an adipose model through environmental control according to an embodiment of the disclosure.

Referring to FIG. 9, the proliferation results of the adipose models created by the conventional methods of mimicking the adipose tissue and the adipose model created using the first bio-ink according to the disclosure were compared.

From the top, the left column of FIG. 9 shows the results of printing the adipose model 1 under the conditions of: 1) 2D culture, 2) Direct printing: Preadipocytes were encapsulated in the AdECM bio-ink and printed without bath suspension, 3) Environmentally uncontrolled in-bath 3D bioprinting (EUIP): Preadipocytes were encapsulated in the AdECM second bio-ink and printed in the environmentally-uncontrolled bath suspension (without alginate), and 4) Environmentally controlled in-bath 3D bioprinting (ECIP): Preadipocytes were encapsulated in the AdECM second bio-ink and printed in the environmentally controlled bath suspension (with alginate).

Referring to the middle column of FIG. 9, adipocytes differentiated to have size similar to that of the actual mature adipocytes (>50 μm) were observed in the adipose model (ECIP) according to the disclosure. On the other hand, in the adipose models according to the other experimental groups, the adipocytes were differentiated to have a much smaller diameter than that according to the disclosure.

Referring to the right column of FIG. 9, it was confirmed that the adipocytes of the adipose model (ECIP) according to the disclosure were differentiated along a printing path, but the adipocytes according to the other experimental groups were sparsely distributed.

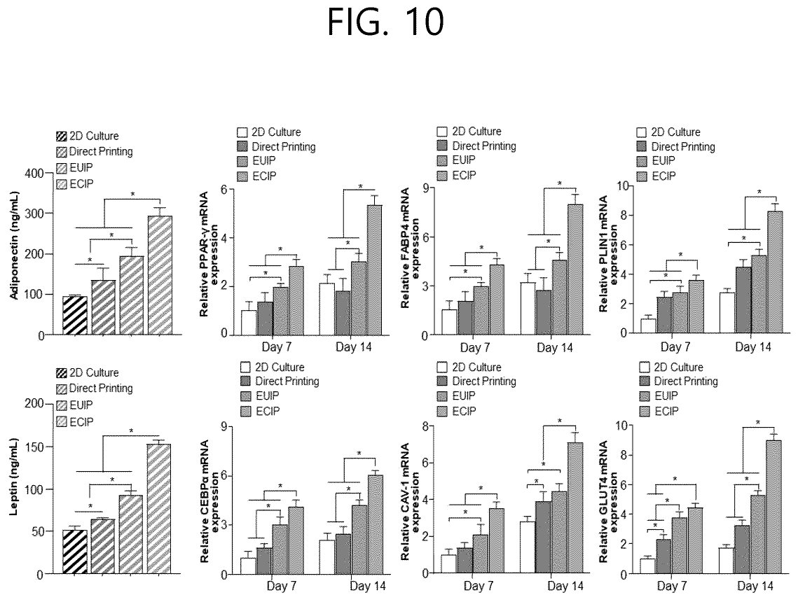

FIG. 10 shows function evaluation results of an adipose model created by a method of producing the adipose mode through environmental control according to an embodiment of the disclosure.

Referring to FIG. 10, the results of subjecting the respective producing methods of FIG. 9 to the quantitative analysis based on the enzyme-linked immunosorbent assay (ELISA) and the polymerase chain reaction (PCR) are shown. As a result of analyzing adiponectin and leptin, i.e., representative hormones secreted by mature adipocytes, through the ELISA, it was confirmed that the adipose model (ECIP) according to the disclosure secreted the largest amount of hormones. This means that many mature adipocytes are distributed in the adiposity model (ECIP) according to the disclosure.

Further, as a result of analyzing genes, which are involved in lipogenesis and droplet genesis, through the PCR, the highest gene expression was observed in the adipose model (ECIP) according to the disclosure, similarly to the ELISA analysis results. This means that cell differentiation and function enhanced as not only cell-matrix interaction but also cell-cell interaction worked closely in the adipose model (ECIP) according to the disclosure.

As described above, with an adipose model producing method based on environmental control, and an adipose model produced by the same, an environment for the adipose model to be three-dimensionally printed is provided to induce the differentiation adipose cells within a limited proliferation area, thereby maximizing the similarity to actual adipose tissue.

1. A method of producing an adipose model through environmental control, the method comprising:
preparing a cell-unfriendly first bio-ink;
forming a bath suspension with the first bio-ink to have a predetermined volume;
preparing a second bio-ink that contains preadipocytes;
producing the adipose model by three-dimensionally printing the second bio-ink in the bath suspension; and
culturing the bath suspension with the adipose model provided therein.

2. The method of claim 1, wherein the first bio-ink contains no cell-binding motifs.

3. The method of claim 2, wherein the first bio-ink contains an adipose derived extracellular matrix.

4. The method of claim 3, wherein the first bio-ink contains a substance to prevent the preadipocytes of the adipose model from infiltrating into the bath suspension during proliferation and differentiation.

5. The method of claim 4, wherein the first bio-ink contains alginate.

6. The method of claim 5, wherein the first bio-ink contains 1 to 2% weight/volume of the adipose derived extracellular matrix.

7. The method of claim 6, wherein the first bio-ink contains 1 to 2% weight/volume of the alginate.

8. The method of claim 7, wherein the bath suspension has shear recovery properties.

9. The method of claim 8, wherein the preadipocytes contained in the second bio-ink have a concentration of 1×10⁶ to 1×10⁸ cells/mL.

10. An adipose model produced using an environmentally-controlled bath suspension, by performing:
preparing a cell-unfriendly first bio-ink;
forming a bath suspension with the first bio-ink to have a predetermined volume;
preparing a second bio-ink that contains preadipocytes;
producing the adipose model by three-dimensionally printing the second bio-ink in the bath suspension; and
culturing the bath suspension with the adipose model provided therein.
